SEQUENCE LISTING (8 sequences)

SEQ ID NO: 1 - protein, 95 aa, Artificial Sequence
  This sequence is synthesized in lab.
KKRIKPIVWPSLPDHKKTLEHLCKKPRKNLNVSFNPESFLDCQIHRVDDIQARDEVEGFLQDTFPQQLEESEKQRLLGSNQEEAYVTMSSFYQNQ

SEQ ID NO: 2 - DNA, 285 bp, Artificial Sequence
  This sequence is synthesized in lab.
aaaaaaaggattaagcctatcgtatggcccagtctccccgatcacaagaagactctggaacacctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttctgggatcaaatcaagaagaagcatatgtcaccatgtccagcttctaccaaaaccag

SEQ ID NO: 3 - protein, 822 aa, Artificial Sequence
  This sequence is synthesized in lab.
QVQLVESGGGVVQPGRSLRLSCAASGFTFSSYGMHWVRQAPGKGLEWVAVIWYDGSNKFYADSVKGRFTISRDNSKNTLYLQMNSLRAEDTAVYYCARRSDYRGYYGMDVWGQGTTVTVSSGSTSGGGSGGGSGGGGSSEIVLTQSPATLSLSPGERATLSCRASQSVINYLAWYQQKPGQAPRLLIYDASNRASGIPARFSGSGSGTDFTLTISSLEPEDFAVYYCQQRRNWPLTFGGGTKVEIKGGGGSGGGGSGGGGSQVQLVQSGAEVKKPGASVKVSCKASGYTFTDFEMHWVRQAPGQGLEWMGDIDPGTGDTAYNLKFKGRVTMTTDTSTSTAYMELRSLRSDDTAVYYCALGAFVYWGQGTLVTVSSGSTSGGGSGGGSGGGGSSDVVMTQSPLSLPVTPGEPASISCRSSQSLANSYGNTYLSWYLQKPGQSPQLLIYGISNRFSGVPDRFSGSGSGTDFTLKISRVEAEDVGVYYCLQGTHQPYTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKKRIKPIVWPSLPDHKKTLEHLCKKPRKNLNVSFNPESFLDCQIHRVDDIQARDEVEGFLQDTFPQQLEESEKQRLLGSNQEEAYVTMSSFYQNQRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 2466 bp, Artificial Sequence
  This sequence is synthesized in lab.
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaattctatgcagactccgtgaagggccgattcaccatctccagagacaattccaagaacacgctgtatctgcaaatgaacagcctgagagccgaggacacggctgtgtattactgtgcgaggagaagtgactacaggggctactacggtatggacgtctggggccaagggaccacggtcaccgtctcctcaggcagtactagcggtggtggctccgggggcggttccggtgggggcggcagcagcgaaattgtgttgacacagtctccagccaccctgtctttgtctccaggggaaagagccaccctctcctgcagggccagtcagagtgttatcaactacttagcctggtaccaacagaaacctggccaggctcccaggctcctcatctatgatgcatccaacagggcctctggcatcccagccaggttcagtggcagtgggtctgggacagacttcactctcaccatcagcagcctagagcctgaagattttgcagtttattactgtcagcagcgtcgcaactggccgctcactttcggcggagggaccaaggtggagatcaaaggaggtggtggatccggaggtggtggctccggaggtggtggatcccaggttcagctggtgcagtctggagctgaggtgaagaagcctggggcctcagtgaaggtctcctgcaaggcttctggttacacctttaccgactttgaaatgcactgggtgcgacaggcccctggacaagggcttgagtggatgggagatattgatcctggaactggtgatactgcctacaatctgaagttcaagggcagagtcaccatgaccacagacacatccacgagcacagcctacatggagctgaggagcctgaggtctgacgacacggccgtgtattactgtgcgttgggggcctttgtttactggggccagggaaccctggtcaccgtctcctcaggcagtactagcggtggtggctccgggggcggttccggtgggggcggcagcagcgatgttgtgatgactcagtctccactctccctgcccgtcacccctggagagccggcctccatctcctgcaggtctagtcagagtcttgcaaacagttatgggaacacctatttgtcttggtacctgcagaagccagggcagtctccacagctcctgatctatgggatttccaacagattttctggggtccctgacaggttcagtggcagtggatcaggcacagattttacactgaaaatcagcagagtggaggctgaggacgttggggtttattactgcttacaaggtacacatcagccgtacacgtttggccaggggaccaagctggagatcaaaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaggagtaagaggagcaggctcctgcacagtgactacatgaacatgactccccgccgccccgggcccacccgcaagcattaccagccctatgccccaccacgcgacttcgcagcctatcgctccaaaaaaaggattaagcctatcgtatggcccagtctccccgatcacaagaagactctggaacacctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttctgggatcaaatcaagaagaagcatatgtcaccatgtccagcttctaccaaaaccagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 5 - DNA, 2181 bp, Artificial Sequence
  This sequence is synthesized in lab.
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaattctatgcagactccgtgaagggccgattcaccatctccagagacaattccaagaacacgctgtatctgcaaatgaacagcctgagagccgaggacacggctgtgtattactgtgcgaggagaagtgactacaggggctactacggtatggacgtctggggccaagggaccacggtcaccgtctcctcaggcagtactagcggtggtggctccgggggcggttccggtgggggcggcagcagcgaaattgtgttgacacagtctccagccaccctgtctttgtctccaggggaaagagccaccctctcctgcagggccagtcagagtgttatcaactacttagcctggtaccaacagaaacctggccaggctcccaggctcctcatctatgatgcatccaacagggcctctggcatcccagccaggttcagtggcagtgggtctgggacagacttcactctcaccatcagcagcctagagcctgaagattttgcagtttattactgtcagcagcgtcgcaactggccgctcactttcggcggagggaccaaggtggagatcaaaggaggtggtggatccggaggtggtggctccggaggtggtggatcccaggttcagctggtgcagtctggagctgaggtgaagaagcctggggcctcagtgaaggtctcctgcaaggcttctggttacacctttaccgactttgaaatgcactgggtgcgacaggcccctggacaagggcttgagtggatgggagatattgatcctggaactggtgatactgcctacaatctgaagttcaagggcagagtcaccatgaccacagacacatccacgagcacagcctacatggagctgaggagcctgaggtctgacgacacggccgtgtattactgtgcgttgggggcctttgtttactggggccagggaaccctggtcaccgtctcctcaggcagtactagcggtggtggctccgggggcggttccggtgggggcggcagcagcgatgttgtgatgactcagtctccactctccctgcccgtcacccctggagagccggcctccatctcctgcaggtctagtcagagtcttgcaaacagttatgggaacacctatttgtcttggtacctgcagaagccagggcagtctccacagctcctgatctatgggatttccaacagattttctggggtccctgacaggttcagtggcagtggatcaggcacagattttacactgaaaatcagcagagtggaggctgaggacgttggggtttattactgcttacaaggtacacatcagccgtacacgtttggccaggggaccaagctggagatcaaaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaggagtaagaggagcaggctcctgcacagtgactacatgaacatgactccccgccgccccgggcccacccgcaagcattaccagccctatgccccaccacgcgacttcgcagcctatcgctcccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 6 - DNA, 2766 bp, Artificial Sequence
  This sequence is synthesized in lab.
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaattctatgcagactccgtgaagggccgattcaccatctccagagacaattccaagaacacgctgtatctgcaaatgaacagcctgagagccgaggacacggctgtgtattactgtgcgaggagaagtgactacaggggctactacggtatggacgtctggggccaagggaccacggtcaccgtctcctcaggcagtactagcggtggtggctccgggggcggttccggtgggggcggcagcagcgaaattgtgttgacacagtctccagccaccctgtctttgtctccaggggaaagagccaccctctcctgcagggccagtcagagtgttatcaactacttagcctggtaccaacagaaacctggccaggctcccaggctcctcatctatgatgcatccaacagggcctctggcatcccagccaggttcagtggcagtgggtctgggacagacttcactctcaccatcagcagcctagagcctgaagattttgcagtttattactgtcagcagcgtcgcaactggccgctcactttcggcggagggaccaaggtggagatcaaaggaggtggtggatccggaggtggtggctccggaggtggtggatcccaggttcagctggtgcagtctggagctgaggtgaagaagcctggggcctcagtgaaggtctcctgcaaggcttctggttacacctttaccgactttgaaatgcactgggtgcgacaggcccctggacaagggcttgagtggatgggagatattgatcctggaactggtgatactgcctacaatctgaagttcaagggcagagtcaccatgaccacagacacatccacgagcacagcctacatggagctgaggagcctgaggtctgacgacacggccgtgtattactgtgcgttgggggcctttgtttactggggccagggaaccctggtcaccgtctcctcaggcagtactagcggtggtggctccgggggcggttccggtgggggcggcagcagcgatgttgtgatgactcagtctccactctccctgcccgtcacccctggagagccggcctccatctcctgcaggtctagtcagagtcttgcaaacagttatgggaacacctatttgtcttggtacctgcagaagccagggcagtctccacagctcctgatctatgggatttccaacagattttctggggtccctgacaggttcagtggcagtggatcaggcacagattttacactgaaaatcagcagagtggaggctgaggacgttggggtttattactgcttacaaggtacacatcagccgtacacgtttggccaggggaccaagctggagatcaaaaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaggagtaagaggagcaggctcctgcacagtgactacatgaacatgactccccgccgccccgggcccacccgcaagcattaccagccctatgccccaccacgcgacttcgcagcctatcgctccaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctccccttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 7 - protein, 727 aa, Artificial Sequence
  This sequence is synthesized in lab.
QVQLVESGGGVVQPGRSLRLSCAASGFTFSSYGMHWVRQAPGKGLEWVAVIWYDGSNKFYADSVKGRFTISRDNSKNTLYLQMNSLRAEDTAVYYCARRSDYRGYYGMDVWGQGTTVTVSSGSTSGGGSGGGSGGGGSSEIVLTQSPATLSLSPGERATLSCRASQSVINYLAWYQQKPGQAPRLLIYDASNRASGIPARFSGSGSGTDFTLTISSLEPEDFAVYYCQQRRNWPLTFGGGTKVEIKGGGGSGGGGSGGGGSQVQLVQSGAEVKKPGASVKVSCKASGYTFTDFEMHWVRQAPGQGLEWMGDIDPGTGDTAYNLKFKGRVTMTTDTSTSTAYMELRSLRSDDTAVYYCALGAFVYWGQGTLVTVSSGSTSGGGSGGGSGGGGSSDVVMTQSPLSLPVTPGEPASISCRSSQSLANSYGNTYLSWYLQKPGQSPQLLIYGISNRFSGVPDRFSGSGSGTDFTLKISRVEAEDVGVYYCLQGTHQPYTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 921 aa, Artificial Sequence
  This sequence is synthesized in lab.
QVQLVESGGGVVQPGRSLRLSCAASGFTFSSYGMHWVRQAPGKGLEWVAVIWYDGSNKFYADSVKGRFTISRDNSKNTLYLQMNSLRAEDTAVYYCARRSDYRGYYGMDVWGQGTTVTVSSGSTSGGGSGGGSGGGGSSEIVLTQSPATLSLSPGERATLSCRASQSVINYLAWYQQKPGQAPRLLIYDASNRASGIPARFSGSGSGTDFTLTISSLEPEDFAVYYCQQRRNWPLTFGGGTKVEIKGGGGSGGGGSGGGGSQVQLVQSGAEVKKPGASVKVSCKASGYTFTDFEMHWVRQAPGQGLEWMGDIDPGTGDTAYNLKFKGRVTMTTDTSTSTAYMELRSLRSDDTAVYYCALGAFVYWGQGTLVTVSSGSTSGGGSGGGSGGGGSSDVVMTQSPLSLPVTPGEPASISCRSSQSLANSYGNTYLSWYLQKPGQSPQLLIYGISNRFSGVPDRFSGSGSGTDFTLKISRVEAEDVGVYYCLQGTHQPYTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSPSRSLDCRESGKNGPHVYQDLLLSLGTTNSTLPPPFSLQSGILTLNPVAQGQPILTSLGSNQEEAYVTMSSFYQNQRVKFSRSADAPAYKQGQNQLYNELNLGRREEYDVLDKRRGRDPEMGGKPRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0052014 A1
(43) Pub. Date: Feb. 15, 2024

(54) TRUNCATED BODY OF IL7Ra AND USE THEREOF IN PREPARATION OF MEDICATION FOR TREATING TUMOR

(71) Applicant: Beijing Neurosurgical Institute, Beijing (CN)

(72) Inventors: Wei ZHANG, Beijing (CN); Tao JIANG, Beijing (CN); You ZHAI, Beijing (CN); Guanzhang LI, Beijing (CN)

(73) Assignee: Beijing Neurosurgical Institute, Beijing (CN)

(21) Appl. No.: 18/019,010

(22) PCT Filed: Jul. 30, 2021
(86) PCT No.: PCT/CN2021/109864

§ 371 Date: Jan. 31, 2023

(30) Foreign Application Priority Data
Jul. 31, 2020 (CN) 202010760960.3

(51) Int. Cl. C07K 14/715; C12N 15/63; C07K 16/28; A61K 39/00

(52) U.S. Cl. CPC C07K 14/7155; C12N 15/63; C07K 16/2896; C07K 16/2884; A61K 39/4631; A61K 39/4611; C07K 2319/03; C07K 2319/02; A61K 2239/13

(57) ABSTRACT

The present disclosure relates to a truncated body of IL7Rα. The amino acid sequence of the truncated body of IL7Rα s a sequence shown in SEQ ID NO. 1. A T cell expressing a chimeric antigen receptor containing the truncated body of IL7Rα can effectively kill a tumor cell.

### CROSS-REFERENCE TO RELATED APPLICATION

This application is a national stage application under 35 U.S.C. 371 of International Application No. PCT/CN2021/109864, filed on Jul. 30, 2021, which claims priority to Chinese Patent Application No. 202010760960.3, filed on Jul. 31, 2020, the entire contents of each are herein wholly incorporated by reference.

The material in the ASCII text file report (inno_13455_20230209_sequence_listing_rev.txt; and Date of Creation: Feb. 9, 2023, having the size of 33,381 bytes, filed for the present application via EFS-web in replacement of electronic sequence listing (inno_13145_20230131_Sequence_listing.txt; Size: 33,382 bytes; and Date of Creation: Jan. 31, 2023), is incorporated by reference in the specification for all purposes. The Sequence listing includes no new matter.

### TECHNICAL FIELD

The disclosure relates to the field of pharmaceutical biotechnology, in particular to a truncated body of IL7Rα, an encoding nucleic acid, an expression vector, a cell, a pharmaceutical composition and use thereof.

### BACKGROUND

Chimeric antigen receptor (CAR) is a synthetic T cell receptor consisting of an antigen binding domain, a transmembrane domain and an intracellular signaling domain. The antigen binding domain is located outside a T cell membrane, includes a single-chain antibody or ligand, and is used for specifically binding to a target antigen. The intracellular signaling domain is located in the T cell membrane, and is used for transmitting signals to a T cell to stimulate the immune response of the T cell.

The CAR can target and identify the target antigen on the surface of a tumor cell, so the T cell expressing the CAR can be used for targeting and killing a tumor cell. However, the existing T cell expressing the CAR is still weak in killing the tumor cell.

### SUMMARY

The objective of the disclosure is to overcome the problem that the existing T cell expressing a chimeric antigen receptor (CAR) is still weak in killing a tumor cell, and to provide a truncated body of IL7Rα.

In order to achieve the above objective, in a first aspect, the disclosure provides a truncated body of IL7Rα, the amino acid sequence of which includes a sequence shown in SEQ ID NO. 1.

In a second aspect, the disclosure provides a nucleic acid encoding the truncated body of IL7Rα according to the first aspect; and preferably, the nucleic acid has a nucleotide sequence shown in SEQ ID NO. 2.

In a third aspect, the disclosure provides a fusion protein containing an antigen binding domain, a transmembrane domain and an intracellular signaling domain that are sequentially linked; the amino acid sequence of the intracellular signaling domain includes the amino acid sequence of the truncated body of IL7Rα according to the first aspect; preferably, the antigen binding domain includes an anti-CD44 single-chain antibody and/or an anti-CD133 single-chain antibody, and the intracellular signaling domain includes the truncated body of IL7Rα; and more preferably, the fusion protein has an amino acid sequence shown in SEQ ID NO. 3.

In a fourth aspect, the disclosure provides a fusion nucleic acid encoding the fusion protein according to the third aspect; and preferably, the fusion nucleic acid has a nucleotide sequence shown in SEQ ID NO. 4.

In a fifth aspect, the disclosure provides an expression vector, the expression vector is inserted with an expression cassette, the expression cassette includes a first nucleic acid fragment encoding an antigen binding molecule and a second nucleic acid fragment encoding an intracellular signaling molecule, the intracellular signaling molecule contains the truncated body of IL7Rα according to the first aspect, and an IRES element or a 2 A peptide encoding sequence is inserted between the first nucleic acid fragment and the second nucleic acid fragment; and preferably, the expression cassette is the fusion nucleic acid according to the fourth aspect.

In a sixth aspect, the disclosure provides a cell expressing a chimeric antigen receptor, the cell expressing a chimeric antigen receptor is obtained by transfection of the expression vector according to the fifth aspect by a host cell, and the chimeric antigen receptor contains the truncated body of IL7Rα according to the first aspect.

Optionally, the host cell is a T cell.

In a seventh aspect, the disclosure provides use of the truncated body of IL7Rα according to the first aspect, the nucleic acid according to the second aspect, the fusion protein according to the third aspect, the fusion nucleic acid according to the fourth aspect, the expression vector according to the fifth aspect, and the cell expressing a chimeric antigen receptor according to the sixth aspect in preparation of a medication for treating a tumor.

Optionally, the tumor is glioma.

In an eighth aspect, the disclosure provides a pharmaceutical composition, an active ingredient of which includes the cell expressing a chimeric antigen receptor containing the truncated body of IL7Rα according to the sixth aspect.

Through the above technical solution, a T cell expressing a chimeric antigen receptor containing the truncated body of IL7Rα provided by the disclosure can effectively kill a tumor cell.

Other features and advantages of the disclosure will be described in detail in the following specific embodiments.

### BRIEF DESCRIPTION OF THE DRAWINGS

The accompanying drawings are intended to provide a further understanding of the disclosure, constitute a part of the description, and are used for interpreting the disclosure together with the following specific embodiments, rather than limiting the disclosure. In the figures:

FIG. 1 is a flow cytometer test result diagram of CAR-T 1 cells according to an embodiment of the disclosure;

FIG. 2 is a flow cytometry test result diagram of CAR-T 2 cells provided by an embodiment of the disclosure;

FIG. 3 is a flow cytometry test result diagram of CAR-T 3 cells provided by an embodiment of the disclosure; and

FIG. 4 is a flow cytometer test result diagram of CAR-T 4 cells according to an embodiment of the disclosure.

### DETAILED DESCRIPTION

The specific embodiments of the disclosure will be described in detail below with reference to the accompanying drawings. It should be understood that the specific embodiments described herein are merely used for illustrating and interpreting the disclosure, rather than limiting the disclosure.

A first aspect of the disclosure provides a truncated body of IL7Rα. The amino acid sequence of the truncated body of IL7Rα includes a sequence shown in SEQ ID NO. 1.

The sequence shown in SEQ ID NO. 1 is as follows:

KKRIKPIVWPSLPDHKKTLEHLCKKPRKNLNVSFNPESFLDCQIHRVDD
IQARDEVEGFLQDTFPQQLEESEKQRLLGSNQEEAYVTMSSFYQNQ.

The inventors of the disclosure found that the above-mentioned truncated body of IL7Rα can effectively prolong the survival time of an activated T cell. Therefore, a T cell expressing a chimeric antigen receptor containing the truncated body of IL7Rα has a long survival time and can effectively kill a tumor cell.

A second aspect of the disclosure provides a nucleic acid encoding the truncated body of IL7Rα according to the first aspect. Preferably, the nucleic acid has a nucleotide sequence shown in SEQ ID NO. 2.

The nucleotide sequence shown in SEQ ID NO. 2 is as follows:

aaaaaaaggattaagcctatcgtatggcccagtctccccgatcacaaga
agactctggaacacctttgtaagaaaccaagaaaaaatttaaatgtgag
tttcaatcctgaaagtttcctggactgccagattcatagggtggatgac
attcaagctagagatgaagtggaaggttttctgcaagatacgtttcctc
agcaactagaagaatctgagaagcagaggcttctgggatcaaatcaaga
agaagcatatgtcaccatgtccagcttctaccaaaaccag.

A third aspect of the disclosure provides a fusion protein containing an antigen binding domain, a transmembrane domain and an intracellular signaling domain that are sequentially linked. The amino acid sequence of the intracellular signaling domain includes the amino acid sequence of the truncated body of IL7Rα according to the first aspect. Preferably, the antigen binding domain includes an anti-CD44 single-chain antibody and/or an anti-CD133 single-chain antibody, and the intracellular signaling domain includes the truncated body of IL7Rα. More preferably, the fusion protein has an amino acid sequence shown in SEQ ID NO. 3.

The fusion protein shown in SEQ ID NO. 3 consists of an anti-CD44 single-chain antibody, an anti-CD133 single-chain antibody, a CD28, a truncated body of IL7Rα, and a CD3.

The amino acid sequence shown in SEQ ID NO. 3 is as follows:

QVQLVESGGGVVQPGRSLRLSCAASGFTFSSYGMHWVRQAPGKGLEWVA
VIWYDGSNKFYADSVKGRFTISRDNSKNTLYLQMNSLRAEDTAVYYCAR
RSDYRGYYGMDVWGQGTTVTVSSGSTSGGGSGGGSGGGGSSEIVLTQSP
ATLSLSPGERATLSCRASQSVINYLAWYQQKPGQAPRLLIYDASNRASG
IPARFSGSGSGTDFTLTISSLEPEDFAVYYCQQRRNWPLTFGGGTKVEI
KGGGGSGGGGSGGGGSQVQLVQSGAEVKKPGASVKVSCKASGYTFTDFE
MHWVRQAPGQGLEWMGDIDPGTGDTAYNLKFKGRVTMTTDTSTSTAYME
LRSLRSDDTAVYYCALGAFVYWGQGTLVTVSSGSTSGGGSGGGSGGGGS
SDVVMTQSPLSLPVTPGEPASISCRSSQSLANSYGNTYLSWYLQKPGQS
PQLLIYGISNRFSGVPDRFSGSGSGTDFTLKISRVEAEDVGVYYCLQGT
HQPYTFGQGTKLEIKTTTPAPRPPTPAPTIASQPLSLRPEACRPAAGGA
VHTRGLDFACDIYIWAPLAGTCGVLLLSLVITLYCRSKRSRLLHSDYMN
MTPRRPGPTRKHYQPYAPPRDFAAYRSKKRIKPIVWPSLPDHKKTLEHL
CKKPRKNLNVSFNPESFLDCQIHRVDDIQARDEVEGFLQDTFPQQLEES
EKQRLLGSNQEEAYVTMSSFYQNQRVKFSRSADAPAYKQGQNQLYNELN
LGRREEYDVLDKRRGRDPEMGGKPRRKNPQEGLYNELQKDKMAEAYSEI
GMKGERRRGKGHDGLYQGLSTATKDTYDALHMQALPPR.

A fourth aspect of the disclosure provides a fusion nucleic acid encoding the fusion protein according to the third aspect. Preferably, the fusion nucleic acid has a nucleotide sequence shown in SEQ ID NO. 4.

The nucleotide sequence shown in SEQ ID NO. 4 is used for encoding the amino acid sequence shown in SEQ ID NO. 3.

The nucleotide sequence shown in SEQ ID NO. 4 is as follows:

caggtgcagctggtggagtctgggggaggcgtggtccagcctgggaggtccctgagactctcctgtgcagcgtctggattc
accttcagtagctatggcatgcactgggtccgccaggctccaggcaaggggctggagtgggtggcagttatatggtatgatggaagtaat
aaattctatgcagactccgtgaagggccgattcaccatctccagagacaattccaagaacacgctgtatctgcaaatgaacagcctgagag
ccgaggacacggctgtgtattactgtgcgaggagaagtgactacaggggctactacggtatggacgtctggggccaagggaccacggt
caccgtctcctcaggcagtactagcggtggtggctccgggggcggttccggtgggggcggcagcagcgaaattgtgttgacacagtctc
cagccaccctgtctttgtctccaggggaaagagccaccctctcctgcagggccagtcagagtgttatcaactacttagcctggtaccaaca
gaaacctggccaggctcccaggctcctcatctatgatgcatccaacagggcctctggcatcccagccaggttcagtggcagtgggtctgg
gacagacttcactctcaccatcagcagcctagagcctgaagattttgcagtttattactgtcagcagcgtcgcaactggccgctcactttcgg
cggagggaccaaggtggagatcaaaggaggtggtggatccggaggtggtggctccggaggtggtggatcccaggttcagctggtgca
gtctggagctgaggtgaagaagcctggggcctcagtgaaggtctcctgcaaggcttctggttacacctttaccgactttgaaatgcactggg
tgcgacaggcccctggacaagggcttgagtggatgggagatattgatcctggaactggtgatactgcctacaatctgaagttcaagggca
gagtcaccatgaccacagacacatccacgagcacagcctacatggagctgaggagcctgaggtctgacgacacggccgtgtattactgt
gcgttgggggcctttgtttactggggccagggaaccctggtcaccgtctcctcaggcagtactagcggtggtggctccgggggcggttcc
ggtgggggcggcagcagcgatgttgtgatgactcagtctccactctccctgcccgtcacccctggagagccggcctccatctcctgcagg
tctagtcagagtcttgcaaacagttatgggaacacctatttgtcttggtacctgcagaagccagggcagtctccacagctcctgatctatggg
atttccaacagattttctggggtccctgacaggttcagtggcagtggatcaggcacagattttacactgaaaatcagcagagtggaggctga
ggacgttggggtttattactgcttacaaggtacacatcagccgtacacgtttggccaggggaccaagctggagatcaaaaccactacccca
gcaccgaggccacccaccccggctcctaccatcgcctcccagcctctgtccctgcgtccggaggcatgtagacccgcagctggtgggg
ccgtgcatacccggggtcttgacttcgcctgcgatatctacatttgggcccctctggctggtacttgcggggtcctgctgctttcactcgtgat
cactctttactgtaggagtaagaggagcaggctcctgcacagtgactacatgaacatgactccccgccgccccgggcccacccgcaagc
attaccagccctatgccccaccacgcgacttcgcagcctatcgctccaaaaaaaggattaagcctatcgtatggcccagtctccccgatca
caagaagactctggaacacctttgtaagaaaccaagaaaaaatttaaatgtgagtttcaatcctgaaagtttcctggactgccagattcatag
ggtggatgacattcaagctagagatgaagtggaaggttttctgcaagatacgtttcctcagcaactagaagaatctgagaagcagaggcttc
tgggatcaaatcaagaagaagcatatgtcaccatgtccagcttctaccaaaaccagcgcgtgaaattcagccgcagcgcagatgctccag
cctacaagcaggggcagaaccagctctacaacgaactcaatcttggtcggagagaggagtacgacgtgctggacaagcggagaggac
gggacccagaaatggggggaagccgcgcagaaagaatccccaagagggcctgtacaacgagctccaaaaggataagatggcagaa
gcctatagcgagattggtatgaaaggggaacgcagaagaggcaaaggccacgacggactgtaccagggactcagcaccgccaccaa
ggacacctatgacgctcttcacatgcaggccctgccgcctcgg.

A fifth aspect of the disclosure provides an expression vector, the expression vector is inserted with an expression cassette, the expression cassette includes a first nucleic acid fragment encoding an antigen binding molecule and a second nucleic acid fragment encoding an intracellular signaling molecule, the intracellular signaling molecule contains the truncated body of IL7Rα according to the first aspect, and an IRES element or a 2 A peptide coding sequence is inserted between the first nucleic acid fragment and the second nucleic acid fragment; and preferably, the expression cassette is the fusion nucleic acid according to the fourth aspect.

A sixth aspect of the disclosure provides a cell expressing a chimeric antigen receptor, the cell expressing a chimeric antigen receptor is obtained by transfection of the expression vector according to the fifth aspect by a host cell, and the chimeric antigen receptor contains the truncated body of IL7Rα according to the first aspect.

Optionally, the host cell is a T cell.

A seventh aspect of the disclosure provides use of the truncated body of IL7Rα according to the first aspect, the nucleic acid according to the second aspect, the fusion protein according to the third aspect, the fusion nucleic acid according to the fourth aspect, the expression vector according to the fifth aspect, and the cell expressing a chimeric antigen receptor according to the sixth aspect in preparation of a medication for treating a tumor.

Optionally, the tumor is glioma.

An eighth aspect of the disclosure provides a pharmaceutical composition, an active ingredient of which includes the cell expressing a chimeric antigen receptor containing the truncated body of IL7Rα according to the sixth aspect.

The disclosure is further described by the following examples, but the disclosure is not limited thereby.

### Example 1

This example was used to explain the construction of expression vectors.
(1) Nucleotide sequences shown in SEQ ID NO. 4, SEQ ID NO. 5 and SEQ ID NO. 6 were respectively synthesized by using a full sequence synthesis method. The nucleotide sequence shown in SEQ ID NO. 4 was used for encoding a fusion protein shown in SEQ ID NO. 3. The nucleotide sequence shown in SEQ ID NO. 5 was used for encoding a fusion protein shown in SEQ ID NO. 7. The nucleotide sequence shown in SEQ ID NO. 6 was used for encoding a fusion protein shown in SEQ ID NO. 8. The compositions of the fusion proteins shown in SEQ ID NO. 3, SEQ ID NO. 7 and SEQ ID NO. 8 were as follows:
G1: the fusion protein shown in SEQ ID NO. 3 consisted of an anti-CD44 single-chain antibody, an anti-CD133 single-chain antibody, a CD28, a truncated body of IL7Rα, and a CD3;
G2: the fusion protein shown in SEQ ID NO. 7 consisted of an anti-CD44 single-chain antibody, an anti-CD133 single-chain antibody, a CD28, and a CD3;
G3: the fusion protein shown in SEQ ID NO. 8 consisted of an anti-CD44 single-chain antibody, an anti-CD133 single-chain antibody, a CD28, an IL7Rα, and a CD3.

The amino acid sequence of the fusion protein shown in SEQ ID NO. 7 was as follows:

QVQLVESGGGVVQPGRSLRLSCAASGFTFSSYGMHWVRQAPGKGLEWVA
VIWYDGSNKFYADSVKGRFTISRDNSKNTLYLQMNSLRAEDTAVYYCAR
RSDYRGYYGMDVWGQGTTVTVSSGSTSGGGSGGGSGGGGSSEIVLTQSP
ATLSLSPGERATLSCRASQSVINYLAWYQQKPGQAPRLLIYDASNRASG
IPARFSGSGSGTDFTLTISSLEPEDFAVYYCQQRRNWPLTFGGGTKVEI
KGGGGSGGGGSGGGGSQVQLVQSGAEVKKPGASVKVSCKASGYTFTDFE
MHWVRQAPGQGLEWMGDIDPGTGDTAYNLKFKGRVTMTTDTSTSTAYME
LRSLRSDDTAVYYCALGAFVYWGQGTLVTVSSGSTSGGGSGGGSGGGGS
SDVVMTQSPLSLPVTPGEPASISCRSSQSLANSYGNTYLSWYLQKPGQS
PQLLIYGISNRFSGVPDRFSGSGSGTDFTLKISRVEAEDVGVYYCLQGT
HQPYTFGQGTKLEIKTTTPAPRPPTPAPTIASQPLSLRPEACRPAAGGA
VHTRGLDFACDIYIWAPLAGTCGVLLLSLVITLYCRSKRSRLLHSDYMN
MTPRRPGPTRKHYQPYAPPRDFAAYRSRVKFSRSADAPAYKQGQNQLYN
ELNLGRREEYDVLDKRRGRDPEMGGKPRRKNPQEGLYNELQKDKMAEAY
SEIGMKGERRRGKGHDGLYQGLSTATKDTYDALHMQALPPR.

The nucleotide sequence shown in SEQ ID NO. 5 was as follows:

caggtgcagctggtggagtctgggggaggcgtggtccagcctgggaggtccctgagactctcctgtgcagcgtctggattc
accttcagtagctatggcatgcactgggtccgccaggctccaggcaaggggctggagtgggtggcagttatatggtatgatggaagtaat
aaattctatgcagactccgtgaagggccgattcaccatctccagagacaattccaagaacacgctgtatctgcaaatgaacagcctgagag
ccgaggacacggctgtgtattactgtgcgaggagaagtgactacaggggctactacggtatggacgtctggggccaagggaccacggt
caccgtctcctcaggcagtactagcggtggtggctccgggggcggttccggtgggggcggcagcagcgaaattgtgttgacacagtctc
cagccaccctgtctttgtctccaggggaaagagccaccctctcctgcagggccagtcagagtgttatcaactacttagcctggtaccaaca
gaaacctggccaggctcccaggctcctcatctatgatgcatccaacagggcctctggcatcccagccaggttcagtggcagtgggtctgg
gacagacttcactctcaccatcagcagcctagagcctgaagattttgcagtttattactgtcagcagcgtcgcaactggccgctcactttcgg
cggagggaccaaggtggagatcaaaggaggtggtggatccggaggtggtggctccggaggtggtggatcccaggttcagctggtgca
gtctggagctgaggtgaagaagcctggggcctcagtgaaggtctcctgcaaggcttctggttacacctttaccgactttgaaatgcactggg
tgcgacaggcccctggacaagggcttgagtggatgggagatattgatcctggaactggtgatactgcctacaatctgaagttcaagggca
gagtcaccatgaccacagacacatccacgagcacagcctacatggagctgaggagcctgaggtctgacgacacggccgtgtattactgt
gcgttgggggcctttgtttactggggccagggaaccctggtcaccgtctcctcaggcagtactagcggtggtggctccgggggcggttcc
ggtgggggcggcagcagcgatgttgtgatgactcagtctccactctccctgcccgtcacccctggagagccggcctccatctcctgcagg
tctagtcagagtcttgcaaacagttatgggaacacctatttgtcttggtacctgcagaagccagggcagtctccacagctcctgatctatggg
atttccaacagattttctggggtccctgacaggttcagtggcagtggatcaggcacagattttacactgaaaatcagcagagtggaggctga
ggacgttggggtttattactgcttacaaggtacacatcagccgtacacgtttggccaggggaccaagctggagatcaaaaccactacccca
gcaccgaggccacccaccccggctcctaccatcgcctcccagcctctgtccctgcgtccggaggcatgtagacccgcagctggtgggg
ccgtgcatacccggggtcttgacttcgcctgcgatatctacatttgggcccctctggctggtacttgcggggtcctgctgctttcactcgtgat
cactctttactgtaggagtaagaggagcaggctcctgcacagtgactacatgaacatgactccccgccgccccgggcccacccgcaagc
attaccagccctatgccccaccacgcgacttcgcagcctatcgctcccgcgtgaaattcagccgcagcgcagatgctccagcctacaagc
aggggcagaaccagctctacaacgaactcaatcttggtcggagagaggagtacgacgtgctggacaagcggagaggacgggaccca
gaaatgggcgggaagccgcgcagaaagaatccccaagagggcctgtacaacgagctccaaaaggataagatggcagaagcctatagc
gagattggtatgaaaggggaacgcagaagaggcaaaggccacgacggactgtaccagggactcagcaccgccaccaaggacacctat
gacgctcttcacatgcaggccctgccgcctcgg.

The amino acid sequence of the fusion protein shown in SEQ ID NO. 8 was as follows:

QVQLVESGGGVVQPGRSLRLSCAASGFTFSSYGMHWVRQAPGKGLEWVA
VIWYDGSNKFYADSVKGRFTISRDNSKNTLYLQMNSLRAEDTAVYYCAR
RSDYRGYYGMDVWGQGTTVTVSSGSTSGGGSGGGSGGGGSSEIVLTQSP
ATLSLSPGERATLSCRASQSVINYLAWYQQKPGQAPRLLIYDASNRASG
IPARFSGSGSGTDFTLTISSLEPEDFAVYYCQQRRNWPLTFGGGTKVEI
KGGGGSGGGGSGGGGSQVQLVQSGAEVKKPGASVKVSCKASGYTFTDFE
MHWVRQAPGQGLEWMGDIDPGTGDTAYNLKFKGRVTMTTDTSTSTAYME
LRSLRSDDTAVYYCALGAFVYWGQGTLVTVSSGSTSGGGSGGGSGGGGS
SDVVMTQSPLSLPVTPGEPASISCRSSQSLANSYGNTYLSWYLQKPGQS
PQLLIYGISNRFSGVPDRFSGSGSGTDFTLKISRVEAEDVGVYYCLQGT
HQPYTFGQGTKLEIKTTTPAPRPPTPAPTIASQPLSLRPEACRPAAGGA
VHTRGLDFACDIYIWAPLAGTCGVLLLSLVITLYCRSKRSRLLHSDYMN
MTPRRPGPTRKHYQPYAPPRDFAAYRSKKRIKPIVWPSLPDHKKTLEHL
CKKPRKNLNVSFNPESFLDCQIHRVDDIQARDEVEGFLQDTFPQQLEES
EKQRLGGDVQSPNCPSEDVVITPESFGRDSSLTCLAGNVSACDAPILSP
SRSLDCRESGKNGPHVYQDLLLSLGTTNSTLPPPFSLQSGILTLNPVAQ
GQPILTSLGSNQEEAYVTMSSFYQNQRVKFSRSADAPAYKQGQNQLYNE
LNLGRREEYDVLDKRRGRDPEMGGKPRKNPQEGLYNELQKDKMAEAYSE
IGMKGERRRGKGHDGLYQGLSTATKDTYDALHMQALPPR.

The nucleotide sequence shown in SEQ ID NO. 6 was as follows:

caggtgcagctggtggagtctgggggaggcgtggtccagcctgggaggtccctgagactctcctgtgcagcgtctggattc
accttcagtagctatggcatgcactgggtccgccaggctccaggcaaggggctggagtgggtggcagttatatggtatgatggaagtaat
aaattctatgcagactccgtgaagggccgattcaccatctccagagacaattccaagaacacgctgtatctgcaaatgaacagcctgagag
ccgaggacacggctgtgtattactgtgcgaggagaagtgactacaggggctactacggtatggacgtctggggccaagggaccacggt
caccgtctcctcaggcagtactagcggtggtggctccgggggcggttccggtgggggcggcagcagcgaaattgtgttgacacagtctc
cagccaccctgtctttgtctccaggggaaagagccaccctctcctgcagggccagtcagagtgttatcaactacttagcctggtaccaaca
gaaacctggccaggctcccaggctcctcatctatgatgcatccaacagggcctctggcatcccagccaggttcagtggcagtgggtctgg
gacagacttcactctcaccatcagcagcctagagcctgaagattttgcagtttattactgtcagcagcgtcgcaactggccgctcactttcgg
cggagggaccaaggtggagatcaaaggaggtggtggatccggaggtggtggctccggaggtggtggatcccaggttcagctggtgca
gtctggagctgaggtgaagaagcctggggcctcagtgaaggtctcctgcaaggcttctggttacacctttaccgactttgaaatgcactggg
tgcgacaggcccctggacaagggcttgagtggatgggagatattgatcctggaactggtgatactgcctacaatctgaagttcaagggca
gagtcaccatgaccacagacacatccacgagcacagcctacatggagctgaggagcctgaggtctgacgacacggccgtgtattactgt
gcgttgggggcctttgtttactggggccagggaaccctggtcaccgtctcctcaggcagtactagcggtggtggctccgggggcggttcc
ggtgggggcggcagcagcgatgttgtgatgactcagtctccactctccctgcccgtcacccctggagagccggcctccatctcctgcagg
tctagtcagagtcttgcaaacagttatgggaacacctatttgtcttggtacctgcagaagccagggcagtctccacagctcctgatctatggg
atttccaacagattttctggggtccctgacaggttcagtggcagtggatcaggcacagattttacactgaaaatcagcagagtggaggctga
ggacgttggggtttattactgcttacaaggtacacatcagccgtacacgtttggccaggggaccaagctggagatcaaaaccactacccca
gcaccgaggccacccaccccggctcctaccatcgcctcccagcctctgtccctgcgtccggaggcatgtagacccgcagctggtgggg
ccgtgcatacccggggtcttgacttcgcctgcgatatctacatttgggcccctctggctggtacttgcggggtcctgctgctttcactcgtgat
cactctttactgtaggagtaagaggagcaggctcctgcacagtgactacatgaacatgactccccgccgccccgggcccacccgcaagc
attaccagccctatgccccaccacgcgacttcgcagcctatcgctccaaaaaaaggattaagcctatcgtatggcccagtctccccgatcat
aagaagactctggaacatctttgtaagaaaccaagaaaaaatttaaatgtgagtttcaatcctgaaagtttcctggactgccagattcataggg
tggatgacattcaagctagagatgaagtggaaggttttctgcaagatacgtttcctcagcaactagaagaatctgagaagcagaggcttgga
ggggatgtgcagagccccaactgcccatctgaggatgtagtcatcactccagaaagctttggaagagattcatccctcacatgcctggctg
ggaatgtcagtgcatgtgacgcccctattctctccccttccaggtccctagactgcagggagagtggcaagaatgggcctcatgtgtacca
ggacctcctgcttagccttgggactacaaacagcacgctgccccctccattttctctccaatctggaatcctgacattgaacccagttgctca
gggtcagcccattcttacttccctgggatcaaatcaagaagaagcatatgtcaccatgtccagcttctaccaaaaccagcgcgtgaaattca
gccgcagcgcagatgctccagcctacaagcaggggcagaaccagctctacaacgaactcaatcttggtcggagagaggagtacgacgt
gctggacaagcggagaggacgggacccagaaatgggcgggaagccgcgcagaaagaatccccaagagggcctgtacaacgagctc
caaaaggataagatggcagaagcctatagcgagattggtatgaaaggggaacgcagaagaggcaaaggccacgacggactgtaccag
ggactcagcaccgccaccaaggacacctatgacgctcttcacatgcaggccctgccgcctcgg.

(2) The three nucleotide sequences synthesized in step (1) were respectively inserted into pLVX-IRES-ΔNGFR (purchased from Clontech Company, article number 631982) as a vector to obtain three kinds of lentivirus expression vectors of this example.

### Example 2

This example was used to explain the preparation and test of T cells expressing a chimeric antigen receptor.
(1) The three kinds of lentivirus expression vectors constructed in Example 1 and an empty pLVX-IRES-ΔNGFR vector were packaged with lentiviruses respectively, and then T cells were cultured in vitro, transfected and proliferated according to the following methods.
(2) T cells in blood were separated according to the following method: 1 mL of sterile PBS and 1 mL of blood were mixed evenly, then slowly added to an upper layer of a lymphocyte separation medium Ficoll, and centrifuged at 4° C. and 400 g for 30 min, with acceleration and deceleration set to 0 respectively. After centrifugation, the upper plasma was removed, the middle white membrane cells were pipetted, PBS was added for re-suspension washing, and centrifugation was carried out for 10 min at 100 g, with normal acceleration and deceleration. After centrifugation, the upper washing solution was removed, 1 mL of 1640+10% FBS+1% double antibody+1×Glutamine medium was added to re-suspend cells, and then the cells were stimulated to proliferate by means of anti-human CD3/CD28 magnetic beads (purchased from Thermo Fisher company), where the concentration of re-suspended cells was 1×10⁶ cells/mL and the amount of magnetic beads added was 100 μL. Then 100 IU/mL rhIL-2 (Peprotech) was added to stimulate culture for 2 days to obtain T cells.
(3) Transfection of lentiviruses was carried out according to the following method: the lentiviruses packaged in step (1) were respectively added to four parts of T cells separated above, and then polybrene with a final concentration of 6 μg/mL was added, followed by uniform mixing and centrifugation at 32° C. and 800 g for 100 min. After centrifugation, the culture was continued in an incubator for 24 h. After the culture, the culture solution was centrifuged at 1500 rpm for 15 min, and the centrifuged cells were inoculated at a density of 1×10⁶/mL into a culture plate and stimulated for culture with rhIL2-100 IU/mL. After that, the medium was changed every 2-3 days until 2-4 weeks, and transfected T lymphocytes CAR-T 1, CAR-T 2, CAR-T 3 and CAR-T 4 were obtained. CAR-T 1 was transfected with the nucleotide sequence shown in SEQ ID NO. 4 and could express the fusion protein shown in SEQ ID NO. 3; CAR-T 2 was transfected with the nucleotide sequence shown in SEQ ID NO. 5 and could express the fusion protein shown in SEQ ID NO. 7; CAR-T 3 was transfected with the nucleotide sequence shown in SEQ ID NO. 6 and could express the fusion protein shown in SEQ ID NO. 8; and CAR-T 4 was transfected with the empty vector.

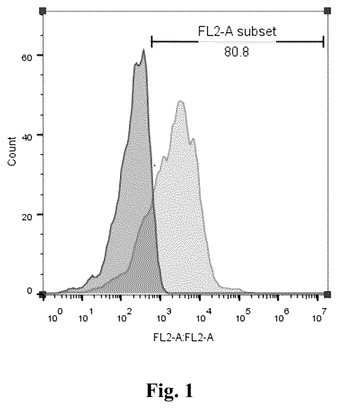

After culture, the cells were re-suspended with PBS, and the ratio of the above four kinds of transfected T lymphocytes and the expression of CAR protein on the surface were tested with a flow cytometer. The test method was as follows: the T cells to be tested after transfection were centrifuged and collected respectively, the supernatant was discarded after the T cells to be tested were washed with PBS once, and a corresponding test amount of monoclonal antibody was added according to antibody instructions and avoided light for 30 min, followed by PBS washing, re-suspension, filtration with a membrane, and sandwich test with a flow cytometer, where the antibody used for the test was a mixture of His-tag labeled CD44 and PE labeled anti-His-tag antibody. The results were shown in FIGS. 1-4.

FIG. 1 is a flow cytometer test result diagram of CAR-T 1 cells according to an embodiment of the disclosure;

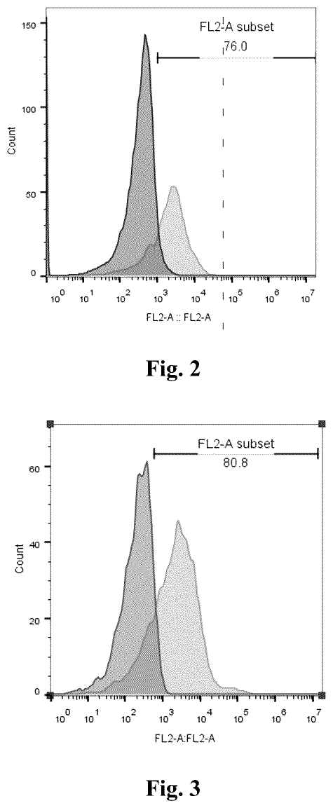

FIG. 2 is a flow cytometry test result diagram of CAR-T 2 cells according to an embodiment of the disclosure;

FIG. 3 is a flow cytometer test result diagram of CAR-T 3 cells according to an embodiment of the disclosure; and

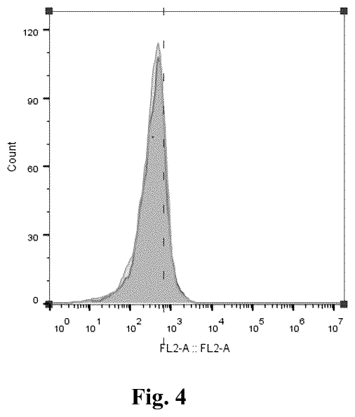

FIG. 4 is a flow cytometer test result diagram of CAR-T 4 cells according to an embodiment of the disclosure.

As can be seen from FIGS. 1-3, other cells different from normal T lymphocytes were detected out from the CAR-T 1 cells, the CAR-T 2 cells and the CAR-T 3 cells. As can be seen from FIG. 4, other cells different from normal T lymphocytes were not detected out from the CAR-T 4 cells. This showed that the CAR-T cells transfected with fusion genes in this example had successfully expressed the target fusion protein.

### Example 3

This example was used to verify the proliferation capacity and survival of the CAR-T cells constructed in Example 2.

Four kinds of CAR-T cells obtained by transfection and culture in Example 2 were respectively mixed with CD44 and CD133 positive glioma stem cells GSC20 according to an effect-target ratio of 10 (the number of T cells: the number of glioma stem cells). The mixed cells were cultured in 24-well plates, where each well contained 10⁵ glioma stem cells, and the reaction system was 1 μL per well. The culture conditions included: 37° C., 5% CO₂, and culture in a saturated humidity incubator. After 0, 4, 8, 12, 18, and 26 days, different CAR-T cells were counted with cell counting plates to explore the proliferation of different CAR-T cells stimulated by target cells.

TABLE 1

| | Proliferation of different CAR-T cells stimulated by target | | | | | |
|---|---|---|---|---|---|---|
| | cells (×10⁶) | | | | | |
| CAR-T cells | Day 0 | Day 4 | Day 8 | Day 12 | Day 18 | Day 26 |
| CAR-T 1 | 1.029 | 2.860 | 3.995 | 4.533 | 5.364 | 3.788 |
| CAR-T 2 | 1.147 | 2.435 | 3.151 | 3.080 | 0.665 | 0 |
| CAR-T 3 | 1.149 | 2.692 | 3.552 | 3.953 | 3.253 | 0 |
| CAR-T 4 | 1.135 | 2.494 | 2.999 | 0.464 | 0 | 0 |

As can be seen from Table 1, the CAR-T cells expressing the above-mentioned truncated body of IL7Rα had stronger proliferation capacity and longer survival.

### Comparative Example

The CAR-T 1 cells obtained by transfection and culture in Example 2 and conventional second generation CAR-T cells (EGFR vIII-CD28-CD3) targeting a classical tumor target EGFR vIII were respectively mixed with CD44 positive and CD133 positive glioma stem cells GSC20 according to different effect-target ratios (the number of T cells: the number of glioma stem cells). The mixed cells were cultured in 96-well plates, where each well contained 4×10⁴ glioma stem cells, and the reaction system was 200 μL per well. The culture conditions included: 37° C., 5% CO₂, and culture in a saturated humidity incubator for 4 h.

Determination of lactate dehydrogenase activity: after centrifugation, 100 μL of supernatant was aspirated from each well and placed in 96-well enzyme-labeled plates, 100 μL of LDH substrate was added to each well, and the reaction was carried out at room temperature for 30 min without light. After the reaction, 50 μL of termination solution was added to each well to terminate the enzymatic reaction. The optical density (OD) was measured at 490 nm with a microplate reader. The average optical density (OD) of each group was calculated, and the lysis rate of glioma stem cells by each kind of T cells was calculated according to the following formula. The results were shown in Table 1.

Lysis rate %=(OD of the experimental group−OD spontaneously released by glioma stem cells−OD naturally released by effector cells)/(maximum OD released by glioma stem cells−OD spontaneously released by glioma stem cells)

The test results were shown in Table 2.

TABLE 2

| | Lysis rate of glioma stem cells by conventional | | | |
|---|---|---|---|---|
| | second generationn CAR-T cells under | | | |
| | different effect-target ratios (%) | | | |
| Effect-target ratio | 5:1 | 3:1 | 2:1 | 1:1 |
| CAR-T 1 | 84.74% | 67.20% | 37.13% | 31.48% |
| Conventional | 17.57% | 14.62% | 13.97% | 11.88% |
| CAR-T | | | | |

As can be seen from Table 2, the ability of the conventional second generation CAR-T cells targeting a classical tumor stem cell target EGFR vIII to kill glioma stem cells was weaker than that of the T cells expressing the truncated body of IL7Rα provided by the disclosure.

The preferred embodiments of the disclosure are described in detail above with reference to the accompanying drawings. However, the disclosure is not limited to the specific details in the above embodiments. Various simple variations may be made to the technical solutions of the disclosure within the scope of the technical idea of the disclosure, and these simple variations fall within the scope of the disclosure.

It should be further noted that the specific technical features described in the above specific embodiments may be combined in any suitable manner without contradiction. In order to avoid unnecessary repetition, the disclosure will not describe various possible combinations.

Moreover, the various different embodiments of the disclosure may be combined randomly without deviating from the idea of the disclosure, and the combinations should also be regarded as the disclosure of the disclosure.

1. A truncated body of IL7Rα, wherein the amino acid sequence of the truncated body of IL7Rα is a sequence shown in SEQ ID NO. 1.

2. A nucleic acid, wherein the nucleic acid encodes the truncated body of IL7Rα according to claim 1.

3. A fusion protein, wherein the fusion protein contains an antigen binding domain, a transmembrane domain and an intracellular signaling domain that are sequentially linked; the amino acid sequence of the intracellular signaling domain comprises the amino acid sequence of the truncated body of IL7Rα according to claim 1.

4. A fusion nucleic acid, wherein the fusion nucleic acid encodes the fusion protein according to claim 3.

5. An expression vector, wherein the expression vector is inserted with an expression cassette, the expression cassette comprises a first nucleic acid fragment encoding an antigen binding molecule and a second nucleic acid fragment encoding an intracellular signaling molecule, the intracellular signaling molecule contains the truncated body of IL7Rα according to claim 1, and an IRES element or a 2 A peptide encoding sequence is inserted between the first nucleic acid fragment and the second nucleic acid fragment.

6. A cell expressing a chimeric antigen receptor, wherein the cell expressing a chimeric antigen receptor is obtained by transfection of the expression vector according to claim 5 by a host cell, and the chimeric antigen receptor contains h truncated body of IL7Rα, wherein the amino acid sequence of the truncated body of IL7Rα is a sequence shown in SEQ ID NO. 1.

7. The cell according to claim 6, wherein the host cell is a T cell.

8-9. (canceled)

10. A pharmaceutical composition, wherein an active ingredient of the pharmaceutical composition comprises the cell expressing a chimeric antigen receptor containing the truncated body of IL7Rα according to claim 6.

11. The nucleic acid according to claim 2, wherein the nucleic acid has a nucleotide sequence shown in SEQ ID NO. 2.

12. The fusion protein according to claim 3, wherein the antigen binding domain comprises an anti-CD44 single-chain antibody and/or an anti-CD133 single-chain antibody, and the intracellular signaling domain comprises the truncated body of IL7Rα.

13. The fusion protein according to claim 3, wherein the fusion protein has an amino acid sequence shown in SEQ ID NO. 3.

14. The fusion nucleic acid according to claim 4, wherein the fusion nucleic acid has a nucleotide sequence shown in SEQ ID NO. 4.

15. The expression vector according to claim 5, wherein the expression cassette is a fusion nucleic acid, wherein the fusion nucleic acid encodes a fusion protein, wherein the fusion protein contains an antigen binding domain, a transmembrane domain and an intracellular signaling domain that are sequentially linked; the amino acid sequence of the intracellular signaling domain comprises the amino acid sequence of a truncated body of IL7Rα, wherein the amino acid sequence of the truncated body of IL7Rα is a sequence shown in SEQ ID NO. 1.
